(19) United States
(12) Patent Application Publication
(10) Pub. No.: US 2010/0234579 A1
(43) Pub. Date: Sep. 16, 2010

(54) SILVER NANOPARTICLE BINDING AGENT CONJUGATES BASED ON MOIETIES WITH TRIPLE CYCLIC DISULFIDE ANCHORING GROUPS

(75) Inventors: Chad A. Mirkin, Wilmette, IL (US); Jae-Seung Lee, Seongbuk-gu (KR); Abigail K.R. Lytton-Jean, Cambridge, MA (US); Sarah J. Hurst, Chicago, IL (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: NORTH WESTERN UNIVERSITY, Evanston (IL)

(21) Appl. No.: 12/599,474

(22) PCT Filed: May 12, 2008
(86) PCT No.: PCT/US08/63441

§ 371 Date: Nov. 9, 2009

Related U.S. Application Data
(60) Provisional application No. 60/917,224, filed on May 10, 2007.

(51) Int. Cl. C07K 16/00; C07H 21/00; C07K 14/00

(52) U.S. Cl. 530/391.1; 536/23.1; 530/400; 977/773

(57) ABSTRACT

The present invention concerns the use of binding agent-functionalized silver nanoparticles for a variety of uses, including molecular diagnostic labels, synthons in programmable materials synthesis approaches, and functional components for nanoelectronic devices. More specifically, the invention provides a new strategy for preparing silver nanoparticle-binding agent conjugates that are based upon moieties with triple cyclic disulfide-anchoring groups.

### STATEMENT OF GOVERNMENT INTEREST

This invention was made with government support under grant number F49620-01-0401, awarded by The Air Force Office of Scientific Research (AFOSR), grant number EEC-0647560, awarded by The National Science Foundation (NSF)/Nanoscale Science and Engineering Centers (NSEC), grant number 5 DP1 OD000285-03, awarded by the National Institute of Health (NIH Pioneer Award), and grant number 5U54 CA U9341-02, awarded by The National Cancer Institute (NCI)/Centers of Cancer Nanotechnology Excellence (CCNE). The government has certain rights in the invention.

### FIELD OF THE INVENTION

The present invention concerns nanoparticles functionalized with binding agents for a variety of uses, including molecular diagnostic labels, synthons in programmable materials synthesis approaches, and functional components for nanoelectronic devices. More specifically, the invention provides a new strategy for preparing silver nanoparticle conjugates that are based upon the utilization of triple cyclic disulfide-anchoring groups.

### BACKGROUND OF THE INVENTION

The discovery and development of DNA-functionalized gold nanoparticle conjugates (DNA-Au NPs) in 1996 (Mirkin et al., Nature 1996, 382, 607-609; Alivisatos et al., Nature 1996, 382, 609-611) has opened up opportunities for fundamental studies of their novel properties (Storhoff et al., J. Am. Chem. Soc. 2000, 122, 4640-4650; Jin et al., J. Am. Chem. Soc. 2003, 125, 1643-1654; Lytton-Jean et al., J. Am. Chem. Soc. 2005, 127, 12754-12755; Lee et al., J. Am. Chem. Soc. 2006, (128), 8899-8903) as well as their application in the assembly of advanced superstructures (Mirkin et al., Nature 1996, 382, 607-609; Alivisatos et al., Nature 1996, 382, 609-611; Niemeyer et al., Eur. J. Inorg. Chem. 2005, 3641-3655), the detection of nucleic acids, proteins, metal ions, and small molecules (Rosi et al., Chem. Rev. 2005, 105, 1547-1562; Nam et al., Science 2003, 301, 1884-1886; Stoeva et al., Angew. Chem. Int. Ed. 2006, 118, 3381-3384; Liu et al., Angew. Chem. Int. Ed. 2006, 45, 90-94; Lee et al., Angew. Chem. Int. Ed. 2007, Early View; Cerruti et al., Anal. Chem. 2006, 78, 3282-3288; He et al., J. Am. Chem. Soc. 2000, 122, 9071-9077; Pavlov et al., J. Am. Chem. Soc. 2004, 126, 11768-11769; Niemeyer, C. M. Angew. Chem. Int. Ed. 2001, 40, 4128-4158; Huang et al., Anal. Chem. 2005, 77, 5735-5741; Su et al., Appl. Phys. Lett. 2003, 82, 3562-3564; Han et al., Angew. Chem. Int. Ed. 2006, 45, 1807-1810; Maxwell et al., J. Am. Chem. Soc. 2002, 124, 9606; Sato et al., J. Am. Chem. Soc. 2003, 125, 8102-8103; Li et al., J. Am. Chem. Soc. 2004, 126, 10958-10961), and as gene silencing agents (Rosi et al., Science 2006, 312, 1027-1030). The utility of DNA-Au NPs is, in part, due to their intense optical, catalytic, and synthetically programmable recognition properties. In addition, when chemically modified in the appropriate manner, they can exhibit highly cooperative binding properties, which are typically characterized by extremely sharp melting transitions (Jin et al., J. Am. Chem. Soc. 2003, 125, 1643-1654). The identification of this cooperativity has led to the development of molecular diagnostic probes that exhibit much higher selectivity and sensitivity for target analytes than conventional molecular fluorophore probes (Rosi et al., Chem. Rev. 2005, 105, 1547-1562; Han et al., Angew. Chem. Int. Ed. 2006, 45, 1807-1810; Storhoff et al., J. Am. Chem. Soc. 1998, 120, 1959-1964; Han et al., J. Am. Chem. Soc. 2006, 128, 4954-4955; Nam et al., J. Am. Chem. Soc. 2004, 126, 5932-59338), and “antisense particle” agents that are significantly more effective at gene knockdown than free DNA based antisense agents (Rosi et al., Science 2006, 312, 1027-1030).

Silver nanoparticles (herein referred to as “Ag NPs”) also have generated significant scientific and technological interest (Cao et al., J. Am. Chem. Soc. 2001, 123, 7961-7962). These particles exhibit higher extinction coefficients relative to gold nanoparticles of the same size, possess a particle size-dependent surface plasmon resonance between ˜390 and 420 nm, are electrochemically and catalytically active, and exhibit Raman enhancement properties (Braun et al., J Am Chem. Soc. 2007 129(20):6378-9; Mulvaney, Langmuir 1996, 12, 788-800; Link et al., J. Phys. Chem. B 1999, 103, 3529-3533; Jiang et al., J. Phys. Chem. B 2005, 109, 1730-1735). As has been extensively demonstrated with gold (Mirkin et al., Nature 1996, 382, 607-609; Jin et al., J. Am. Chem. Soc. 2003, 125, 1643-1654; Hurst et al., Anal. Chem. 2006, 78, 8313-8318; Demers et al., Anal. Chem. 2000, 72, 5535-5541), a common method used to functionalize the surface of noble metals is the adsorption of thiol-containing molecules. However, there have been only a few reports of thiol-functionalized Ag NPs (Tokareva et al., J. Am. Chem. Soc. 2004, 126, 15784-15789; Vidal et al., New. J. Chem. 2005, 28, 812-816), and of the structures prepared, all: (1) show limited stability in saline buffer (up to 0.3 M salt concentration), (2) typically require lengthy synthetic procedures (more than 2 days), and (3) do not exhibit highly cooperative binding as determined by melting analyses (the melting transitions for the hybridized particle aggregates span≧10° C.). Moreover, the possible oligonucleotides that can be used to stabilize the particles are limited with respect to sequence (e.g. poly adenine (A) sequences). (Tokareva et al., J. Am. Chem. Soc. 2004, 126, 15784-15789; Vidal et al., New. J. Chem. 2005, 28, 812-816). These limitations are primarily due to the chemical degradation of the Ag NPs under the functionalization conditions and the susceptibility of the silver surface to oxidation (Cao et al., J. Am. Chem. Soc. 2001, 123, 7961-7962; Yin et al., J. Mater. Chem. 2002, 12, 522-527).

As a result of these limitations, alternative approaches have been developed to enable the conjugation of binding agents to Ag NPs. Attempts to modify the Ag NP surface with more tailorable and robust materials such as gold, silica, or polymers have been considered (Cao et al., J. Am. Chem. Soc. 2001, 123, 7961-7962; Liu et al., Anal. Chem. 2005, 77, 2595-2600; Quaroni et al., J. Am. Chem. Soc. 1999, 121, 10642-10643; Chen et al., Chem. Commun. 2004, 2804-2805). However, they require additional cumbersome chemical modification steps. There thus remains a need in the art for new methods of functionalizing Ag NPs with a binding agent.

### SUMMARY OF THE INVENTION

Provided herein is a silver nanoparticle-binding agent conjugate comprising one or more binding agent, each binding agent attached covalently to a silver nanoparticle through a triple cyclic disulfide functional group moiety.

In one embodiment, the nanoparticle is functionalized with an oligonucleotide which is sufficiently complementary to a target nucleic acid, the nanoparticle through the functionalized oligonucleotide thereby capable of hybridizing to the target nucleic acid forming a nanoparticle/oligonucleotide conjugate/target complex. In one aspect, this nanoparticle/oligonucleotide conjugate/target complex exhibits a sharper melting profile and higher stability, relative to a melting profile and stability of an analogous complex formed with the nucleic acid target and a free oligonucleotide having a sequence identical to the sequence of an oligonucleotide bound to the nanoparticle.

In another embodiment, the binding agent covalently attached to the silver nanoparticle through a triple cyclic disulfide moiety is an antibody.

In a further embodiment, the binding agent that may be covalently attached to the silver nanoparticle through a triple cyclic disulfide moiety is a peptide.

In various aspects, the silver nanoparticle has a diameter that is between about 10 nm and about 100 nm, inclusive.

In an embodiment, the triple cyclic disulfide functional group moiety comprises a cyclic disulfide-containing phosphate derivative.

In another embodiment, the silver nanoparticle-binding agent conjugate further comprises one or more additional cyclic disulfide functional group moieties.

In a further embodiment, a silver nanoparticle-binding agent conjugate is provided wherein the binding agent comprises a spacer portion and a binding portion, the spacer portion being bound to the nanoparticle and comprising the triple cyclic disulfide functional group moiety through which the binding agent is bound to the nanoparticle. In one aspect, the spacer portion comprises from about 1 nucleotide to about 10 nucleotides, inclusive. In another aspect, the spacer portion comprises from about 10 to about 30 nucleotides, inclusive. In still other aspects, the spacer portion comprises all adenine bases, all thymine bases, all cytosine bases, all uracil bases or all guanine bases.

In another embodiment of the invention, a method is provided to functionalize silver nanoparticles with a binding agent, wherein the binding agent is modified with three cyclic disulfide functional groups.

In one aspect, the method for functionalizing a binding agent to a silver nanoparticle to produce a nanoparticle-binding agent conjugate, comprises the steps of contacting (i) a binding agent having a triple cyclic disulfide functional group moiety covalently bound thereto, and (ii) a silver nanoparticle under conditions effective to allow the binding agent to attach to the nanoparticle through the triple cyclic disulfide functional group moiety to produce the nanoparticle-binding agent conjugate. In one aspect, the method comprises attaching at least two binding agents to the nanoparticle wherein the two binding agents bind to different target binding partners. In another aspect of the method, a binding agent attached to the nanoparticle has the ability to bind under appropriate conditions to a target binding partner attached to a second nanoparticle to form an aggregate.

The invention also provides methods of detecting a target nucleic acid. In one embodiment, the method comprising the step of contacting the target nucleic acid with a silver nanoparticle having an oligonucleotide attached thereto under conditions that permit formation of a nanoparticle/oligonucleotide/target nucleic acid complex through hybridization of the oligonucleotide and the target nucleic acid. In one aspect, the oligonucleotide attached to the nanoparticle has a sequence complementary to all or portions of the sequences of the target nucleic acid. In one aspect, formation of the nanoparticle/oligonucleotide/target nucleic acid complex results in a detectable change.

Further aspects of the invention will become apparent from the detailed description provided below. However, it should be understood that the following detailed description and examples, while indicating preferred embodiments of the invention, are given by way of illustration only since various changes and modifications within the spirit and scope of the invention will become apparent to those skilled in the art from this detailed description.

### DESCRIPTION OF THE DRAWINGS

FIG. 1A depicts a schematic illustration of the hybridization of two complementary DNA-Ag NPs, as well as the structure of the disulfide-containing phosphoramidite (DTPA) (FIG. 1B).

FIG. 2 depicts the melting transition for the DNA-Ag NP aggregates.

FIG. 3 depicts melting transitions repeatedly measured for the same DNA-Ag NP aggregates.

FIG. 4A depicts the melting transitions for DNA-Ag NP aggregates (30 nm in diameter) at various salt concentrations, as well as the Tₘ at various salt concentrations (FIG. 4B).

### DETAILED DESCRIPTION OF THE INVENTION

It has been previously reported that multiple thiol groups increase the binding affinity of an oligonucleotide for the surface of a silver nanoparticle (Ag NP), and this binding affinity results in nanoparticle probes with higher stabilities (Li et al., Nucleic Acids Res. 2002, 30, 1558-1562). Others have demonstrated that polydentate ligands often form substantially more stable metal-oligonucleotide complexes than monodentate ligands (Cotton et al., Advanced Inorganic Chemistry. 6th ed.; John Wiley & Sons: New York, 1999; pp 27-29). Therefore, utilization of multiple anchoring groups other than thiols on the oligonucleotide might provide Ag NP oligonucleotide conjugates with a higher degree of stability. It is also known in the art that an oligonucleotide containing a single cyclic disulfide anchoring group binds readily to Ag NPs with higher affinity than monothiol or acyclic disulfide groups (Letsinger et al., Bioconjugate Chem. 2000, 11, 289-291; U.S. Pat. No. 6,767,702).

Against this background, a silver nanoparticle is provided functionalized with a binding agent containing three cyclic disulfide moieties through which the binding agent is attached to the silver nanoparticle. Use of the triple cyclic disulfide functional group moieties to attach a binding agent to a silver nanoparticle has the advantage of producing a silver nanoparticle-binding agent conjugate with unexpectedly high stability and which produces an extremely sharp association/dissociation profile with a target molecule under changing local conditions. Methods of preparing the functionalized silver nanoparticle are also provided.

As used herein, the term “binding agent” means a molecule which is capable of recognizing and associating with one or more specific target molecules. In one aspect, binding agents encompass polynucleotides or polypeptides, including fragment thereof that associate with one or more targets of interest. In another aspect, a binding agent is a small molecules with one or more specific binding properties. The worker of ordinary skill will understand that any member of a specific binding pair of molecules can be a binding agent of the invention, with either member of the pair being attached to a nanoparticle as described herein. Any of a variety of binding agent-binding target, i.e., a binding pair, interactions may be utilized according to methods of the invention, including without limitation biotin/avidin, ligand/receptor, enzyme/substrate, nucleic acid/nucleic acid binding protein, lipid/lipid, lipid/lipid binding protein interactions, hormones-hormone receptors, IgG-protein A, as well as fragments of these binding pairs which maintain affinity for its specific binding partner or partners.

As used herein, the term “triple cyclic disulfide functional group moiety” means cyclic disulfide molecules that have 3, 4, 5, 6 or more atoms in their rings, including the two sulfur atoms. In one aspect and without limitation, the triple cyclic disulfide has a hydrocarbon moiety attached to each cyclic disulfide or is comprised of three disulfide-containing phosphoramidite units (DTPA). In another aspect, the triple cyclic disulfide functional group moiety is present on either terminus of the binding agent.

As used herein, “stable” means that, for a period of at least six months after the conjugates are made, a majority of the oligonucleotides remain attached to the nanoparticles and the oligonucleotides are able to hybridize with nucleic acid and oligonucleotide targets under standard conditions encountered in methods of detecting nucleic acid and methods of nanofabrication.

As used herein, the phrase “nanoparticle having a binding agent attached thereto” refers to a “nanoparticle-binding agent conjugate” or, when utilized in a detection method of the invention, “nanoparticle-binding agent probes,” “nanoparticle probes,” or just “probes.”

The term “melts” is understood in the art to mean a specific dissociation reaction wherein hybridized polynucleotides dissociate, generally brought about by changes in local environmental conditions. In one aspect, the local change is an increase in temperature above a “melting temperature, Tₘ” at which two specific nucleic acids that are hybridized are dissociated by 50%. Changes in local environmental conditions can alter the Tₘ for any en hybridized nucleic acids. While the term “melts” is used herein to describe dissociation of hybridized nucleic acids, it will readily be appreciated that dissociation of the interaction between any two other types of binding pair molecules is referred to simply as “dissociation” and this dissociation is, like melting, affected by local environmental conditions at the site of binding between the binding pair.

The melting properties of nanoparticle-oligonucleotide aggregates are affected by a number of factors, including oligonucleotide surface density, nanoparticle size, interparticle distance, and salt concentration. As with native DNA, the Tₘ of these oligonucleotide-linked nanoparticle structures increases with increasing salt concentration. However, changes in salt concentration do not substantially affect the sharpness of the transition. The sharp salt-induced melting of the nanoparticle-oligonucleotide system, which is not observed in unmodified oligonucleotides of the same sequence, allows one to readily discriminate between perfectly complementary targets and single-base mismatched strands and, thus, to develop high selectivity detection assays and potentially eliminate the need for thermal stringency. There also is a strong dependence of Tₘ on interparticle distance; in general, Tₘ increases with increasing interparticle distance for the DNA-linked nanoparticle aggregates due to less electrostatic/steric repulsion and hence stabilization of the duplex interconnects (Jin et al., 2003, J. Am. Chem. Soc. 125: 1643-1654).

It is to be noted that the term “a” or “an” entity refers to one or more of that entity. For example, “a characteristic” refers to one or more characteristics or at least one characteristic. As such, the terms “a” (or “an”), “one or more” and “at least one” are used interchangeably herein. It is also to be noted that the terms “comprising”, “including”, and “having” have been used interchangeably.

### Nanoparticles

Nanoparticles are thus provided which are functionalized to have a binding agent attached thereto. The size, shape and chemical composition of the nanoparticles contribute to the properties of the resulting binding agent-functionalized nanoparticle. These properties include for example, optical properties, optoelectronic properties, electrochemical properties, electronic properties, stability in various solutions, magnetic properties, and pore and channel size variation. Mixtures of nanoparticles having different sizes, shapes and/or chemical compositions, as well as the use of nanoparticles having uniform sizes, shapes and chemical composition, and therefore a mixture of properties are contemplated. Examples of suitable particles include, without limitation, aggregate particles, isotropic (such as spherical particles), anisotropic particles (such as non-spherical rods, tetrahedral, and/or prisms) and core-shell particles, such as those described in U.S. Pat. No. 7,238,472 and International Publication No. WO 2003/08539, the disclosures of which are incorporated by reference in their entirety.

In one embodiment, the nanoparticle is metallic, and in various aspects, the nanoparticle is a colloidal metal. Thus, in various embodiments, nanoparticles of the invention include metal (including for example and without limitation, silver, gold, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials.

Also, as described in U.S. Patent Publication No 2003/0147966, nanoparticles of the invention include those that are available commercially, as well as those that are synthesized, e.g., produced from progressive nucleation in solution (e.g., by colloid reaction) or by various physical and chemical vapor deposition processes, such as sputter deposition. See, e.g., HaVashi, Vac. Sci. Technol. A5(4):1375-84 (1987); Hayashi, Physics Today, 44-60 (1987); MRS Bulletin, January 1990, 16-47. As further described in U.S. Patent Publication No 2003/0147966, nanoparticles contemplated are alternatively produced using HAuCl₄ and a citrate-reducing agent, using methods known in the art. See, e.g., Marinakos et al., Adv. Mater. 11:34-37 (1999); Marinakos et al., Chem. Mater. 10: 1214-19 (1998); Enustun & Turkevich, J. Am. Chem. Soc. 85: 3317 (1963).

Nanoparticles can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or the amount of surface area that can be derivatized as described herein.

Each nanoparticle utilized in the methods provided has a plurality of oligonucleotides attached to it. In one aspect, methods are provided wherein each nanoparticle is modified with identical oligonucleotides, i.e., each oligonucleotide attached to the nanoparticle has the same length and the same sequence. In other aspects, each nanoparticle is modified with two or more oligonucleotides which are not identical, i.e., at least one of the attached oligonucleotides differ from at least one other attached oligonucleotide in that it has a different length and/or a different sequence.

In instances wherein the target nucleic acid is functionalized on a second, distinct nanoparticle, an oligonucleotide on one nanoparticle hybridizes with the target nucleic acid on the second nanoparticle forming an aggregate.

### Oligonucleotide Attachment to Nanoparticle

Nanoparticles with an oligonucleotides attached thereto are thus provided wherein an oligonucleotide having a triple cyclic disulfide covalently bound to its 5′ or 3′ ends through which the oligonucleotide is attached to the nanoparticle.

In one aspect, nanoparticles are provided wherein the packing density of the oligonucleotides on the surface of the nanoparticle is sufficient to result in cooperative behavior between nanoparticles and between polynucleotide strands on a single nanoparticle. In another aspect, the cooperative behavior between the nanoparticles increases the resistance of the oligonucleotide to degradation. A surface density adequate to make the nanoparticles stable and the conditions necessary to obtain it for a desired combination of nanoparticles and oligonucleotides can be determined empirically. Generally, a surface density of at least 10 pmoles/cm² will be adequate to provide stable nanoparticle-oligonucleotide conjugates. Preferably, the surface density is at least 15 pmoles/cm². Since the ability of the oligonucleotides of the conjugates to hybridize with nucleic acid and oligonucleotide targets can be diminished if the surface density is too great, the surface density is preferably no greater than about 35-40 pmoles/cm². Methods are also provided wherein the oligonucleotide is bound to the nanoparticle at a surface density of at least 10 pmol/cm², at least 15 pmol/cm², at least 20 pmol/cm², at least 25 pmol/cm², at least 30 pmol/cm², at least 35 pmol/cm², at least 40 pmol/cm², at least 45 pmol/cm², at least 50 pmol/cm², or 50 pmol/cm² or more.

The invention provides a method of attaching oligonucleotides to nanoparticles by means of a linker comprising a triple cyclic disulfide functional group moiety. Suitable cyclic disulfides are available commercially (See also, e.g., U.S. Pat. No. 6,767,702). The reduced form of the cyclic disulfides can also be used. In some embodiments, the linker may further comprise a cyclic disulfide-containing phosphate derivative (DSP). In one embodiment, the DSP is produced using a disulfide-containing phosphoramidite unit (DTPA). Methods for synthesizing cyclic disulfides useful for attaching oligonucleotides, and other binding agents as described herein, to nanoparticles are known in the art (See generally, Zoller et al., 2000, Tetrahedron Letters 41:9989-9992, the disclosure of which is incorporated herein by reference in its entirety).

In other embodiments, the linker may further comprise a hydrocarbon moiety attached to the cyclic disulfide. Suitable hydrocarbons are available commercially, and are attached to the cyclic disulfides. Preferably the hydrocarbon moiety is a steroid residue. The linkers are attached to the oligonucleotides and the oligonucleotide-linkers are attached to nanoparticles as described herein.

In some embodiments, the steroid used in the methods of the invention is epiandrosterone, due to its availability, easily derivatized keto alcohol and, as a substituent with a large hydrophobic surface, might be expected to help screen the approach of water soluble molecules to the nanoparticle surface.

Oligonucleotide-nanoparticle conjugates prepared using linkers comprising a steroid residue attached to a cyclic disulfide have unexpectedly been found to be remarkably stable to thiols as compared to conjugates prepared using alkanethiols or acyclic disulfides as the linker (See U.S. Pat. No. 6,767,702). Without being bound by theory, this unexpected stability may be due to the fact that each oligonucleotide is anchored to a nanoparticle through two sulfur atoms per cyclic disulfide functional group moiety, rather than just a single sulfur atom. In particular, it is thought that two adjacent sulfur atoms of each cyclic disulfide would have a chelation effect which would be advantageous in stabilizing the oligonucleotide-nanoparticle conjugates. The large hydrophobic steroid residues of the linkers also appear to contribute to the stability of the conjugates by screening the nanoparticles from the approach of water-soluble molecules to the surfaces of the nanoparticles.

In view of the foregoing, the two sulfur atoms of the cyclic disulfide should preferably be close enough together so that both of the sulfur atoms can attach simultaneously to the nanoparticle. Most preferably, the two sulfur atoms are adjacent each other. Also, the hydrocarbon moiety should be large so as to present a large hydrophobic surface screening the surfaces of the nanoparticles.

Previous methods used to conjugate an oligonucleotide to a nanoparticle via an alkyl mono-thiol or an acyclic disulfide linker resulted in problems when the nanoparticle probes were to be used in a solution containing a thiol, as for example, a PCR solution that contains dithiothreitol (DTT) as a stabilizer for the polymerase enzyme. The surprising stability of the resulting oligonucleotide-nanoparticle conjugates of the invention to thiols described above allows them to be used directly in PCR solutions. Thus, oligonucleotide-nanoparticle conjugates of the invention added as probes to a DNA target to be amplified by PCR can be carried through the 30 or 40 heating-cooling cycles of the PCR and are still able to detect the amplicons without opening the tubes. Opening the sample tubes for addition of probes after PCR can cause serious problems through contamination of the equipment to be used for subsequent tests.

### Oligonucleotides

In aspects of the invention wherein the binding agent is an oligonucleotide, the oligonucleotide is functionalized with a triple cyclic disulfide. As disclosed herein, the triple cyclic disulfide has 3, 4, 5, 6 or more atoms in a ring structure, including the two sulfur atoms per cyclic disulfide. Suitable cyclic disulfides are available commercially or may be synthesized by known procedures. The reduced form of the cyclic disulfides can also be used.

As used herein, the term “oligonucleotide” refers to a single-stranded oligonucleotide having natural and/or unnatural nucleotides. In various aspects, the oligonucleotide is a DNA oligonucleotide, an RNA oligonucleotide, or a modified form of either a DNA oligonucleotide or an RNA oligonucleotide. Oligonucleotides contemplated include those having a specific sequence, as well as those including multiple copies of a single sequence in tandem, for example, two, three, four, five, six, seven, eight, nine, ten or more tandem repeats.

The term “nucleotide” or its plural as used herein is interchangeable with modified forms as discussed herein and otherwise known in the art. In certain instances, the art uses the term “nucleobase” which embraces naturally-occurring nucleotides as well as modifications of nucleotides that can be polymerized. Thus, nucleotide or nucleobase means the naturally occurring nucleobases adenine (A), guanine (G), cytosine (C), thymine (T) and uracil (U) as well as non-naturally occurring nucleobases such as xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Oligonucleotides may also include modified nucleobases. A “modified base” is understood in the art to be one that can pair with a natural base (e.g., adenine, guanine, cytosine, uracil, and/or thymine) and/or can pair with a non-naturally occurring base. Exemplary modified bases are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]-benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. No. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making oligonucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically. Non-naturally occurring nucleobases can be incorporated into the oligonucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Nanoparticles provided that are functionalized with an oligonucleotide, or modified form thereof, generally comprise an oligonucleotide from about 5 nucleotides to about 100 nucleotides in length. More specifically, nanoparticles are functionalized with oligonucleotide that are about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all oligonucleotides intermediate in length of the sizes specifically disclosed to the extent that the oligonucleotide is able to achieve the desired result. Accordingly, oligonucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 nucleotides in length are contemplated.

Oligonucleotides contemplated for attachment to a nanoparticle include those which modulate expression of a gene product expressed from a target polynucleotide. Accordingly, antisense oligonucleotides which hybridize to a target polynucleotide and inhibit translation, siRNA oligonucleotides which hybridize to a target polynucleotide and initiate an RNAse activity (for example RNAse H), triple helix forming oligonucleotides which hybridize to double-stranded polynucleotides and inhibit transcription, and ribozymes which hybridize to a target polynucleotide and inhibit translation, are contemplated.

In various aspects, if a specific mRNA is targeted, a single nanoparticle-binding agent conjugate has the ability to bind to multiple copies of the same transcript. In one aspect, a nanoparticle is provided that is functionalized with identical oligonucleotides, i.e., each oligonucleotide has the same length and the same sequence. In other aspects, the nanoparticle is functionalized with two or more oligonucleotides which are not identical, i.e., at least one of the attached oligonucleotides differ from at least one other attached oligonucleotide in that it has a different length and/or a different sequence. In aspects wherein different oligonucleotides are attached to the nanoparticle, these different oligonucleotides bind to the same single target polynucleotide but at different locations, or bind to different target polynucleotides which encode different gene products Accordingly, in various aspects, a single nanoparticle-binding agent conjugate target more than one gene product. Oligonucleotides are thus target specific polynucleotides, whether at one or more specific regions in the target polynucleotide, or over the entire length of the target polynucleotide as the need may be to effect a desired level of inhibition of gene expression.

In some aspects, oligonucleotides are selected from a library. Preparation of libraries of this type is well know in the art. See, for example, Oligonucleotide libraries: United States Patent Application 20050214782, published Sep. 29, 2005.

### Modified Oligonucleotides

Modified oligonucleotides are contemplated for functionalizing nanoparticles wherein both one or more sugar and/or one or more internucleotide linkage of the nucleotide units in the oligonucleotide is replaced with “non-naturally occurring” groups. In one aspect, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

Other linkages between nucleotides and unnatural nucleotides contemplated for the disclosed oligonucleotides include those described in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920; U.S. Patent Publication No. 20040219565; International Patent Publication Nos. WO 98/39352 and WO 99/14226; Mesmaeker et. al., Current Opinion in Structural Biology 5:343-355 (1995) and Susan M. Freier and Karl-Heinz Altmann, Nucleic Acids Research, 25:4429-4443 (1997), the disclosures of which are incorporated herein by reference.

Specific examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide.”

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated.

Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In still other embodiments, oligonucleotides are provided with phosphorothioate backbones and oligonucleotides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In various forms, the linkage between two successive monomers in the oligo consists of 2 to 4, desirably 3, groups/atoms selected from —CH₂—, —O—, —S—, —NRH—, >C═O, >C═NRH, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHRH)—, where RH is selected from hydrogen and C1-4-alkyl, and R″ is selected from C1-6-alkyl and phenyl. Illustrative examples of such linkages are —CH₂—CH₂—CH₂, —CH₂—CO—CH₂—, —CH₂—CHOH—CH₂—, —O—CH2-O—, —O—CH2-CH2-, —O—CH2-CH═(including R5 when used as a linkage to a succeeding monomer), —CH₂—CH₂—O—, —NRH—CH₂—CH₂—, —CH₂—CH₂—NRH—, —CH₂—NRH—CH₂—, —O—CH₂—CH₂—NRH—, —NRH—CO—O—, —NRH—CO—NRH—, —NRH—CS—NRH—, —NRH—C(═NRH)—NRH—, —NRH—CO—CH₂—NRH—O—CO—O—, —O—CO—CH₂—O—, —O—CH₂—CO—O—, —CH₂—CO—NRH—, —O—CO—NRH—, —NRH—CO—CH₂—, —O—CH₂—CO—NRH—, —O—CH₂—CH₂NRH—, —CH═N—O—, —CH₂—NRH—O—, —CH₂—O—N═ (including R5 when used as a linkage to a succeeding monomer), —CH₂—O—NRH—, —CO—NRH—CH₂—, —CH₂—NRH—O—, —CH₂—NRH—CO—, —O—NRH—CH₂—, —O—NRH, —O—CH₂—S—, —S—CH₂—O—, —CH₂—CH₂—S—, —O—CH₂—CH₂—S—, —S—CH₂—CH═ (including R5 when used as a linkage to a succeeding monomer), —S—CH₂—CH₂—, —S—CH₂—CH₂—O—, —S—CH₂—CH₂S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NRH—, —NRH—S(O)₂—CH₂—; —O—S(O)₂—CH₂—, —O—P(O)₂—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃) —O—, —O—PO(OCH₂CH₂S—R)—O—, —O—PO(BH₃)—O—, —O—PO(NHRN)—O—, —O—P(O)₂—NRH—H—, —NRH—P(O)₂—O—, —O—P(O,NRH)—O—, —CH₂—P(O)₂—O—, —O—P(O)₂—CH₂—, and —O—Si(R″)₂—O—; among which —CH₂—CO—NRH—, —CH₂—NRH—O—, —S—CH₂—O—, —O—P(O)₂—O—P(—O,S)—O—, —O—P(S)₂—O—, —NRHP(O)₂—O—, —O—P(O,NRH)—O—, —O—PO(R″)—O—, —O—PO(CH₃)—O—, and —O—PO(NHRN)—O—, where RH is selected form hydrogen and C1-4-alkyl, and R″ is selected from C1-6-alkyl and phenyl, are contemplated. Further illustrative examples are given in Mesmaeker et. al., 1995, Current Opinion in Structural Biology, 5: 343-355 and Susan M. Freier and Karl-Heinz Altmann, 1997, Nucleic Acids Research, vol 25: pp 4429-4443.

Still other modified forms of oligonucleotides are described in detail in U.S. Patent Application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH2)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In one aspect, a modification includes 2′-methoxyethoxy (2′-O—CH₂CH₂OCH₃, also known as 2′-O-(2-methoxyethyl) or 2′-MOE) (Martin et al., 1995, Helv. Chim. Acta, 78: 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects a methylene (—CH₂—)ₙ group bridging the 2′ oxygen atom and the 4′ carbon atom wherein n is 1 or 2. LNAs and preparation thereof are described in WO 98/39352 and WO 99/14226, the disclosures of which are incorporated herein by reference.

### Oligonucleotide Sequences and Hybridization Methods

In aspects of the invention wherein the functionalized binding agent is an oligonucleotide, the invention provides methods of targeting specific nucleic acids. Any type of nucleic acid may be targeted, and the methods may be used, e.g., for the diagnosis of disease (See, e.g., U.S. Pat. No. 6,767,702), for therapeutic modulation of gene expression (See, e.g., PCT/US2006/022325, the disclosure of which is incorporated herein by reference), for drug delivery (See, e.g., PCT/US2006/022325), and in sequencing of nucleic acids (See, e.g., U.S. Pat. No. 6,878,814, the disclosure of which is incorporated herein by reference). Examples of nucleic acids that can be targeted by the methods of the invention include genes (e.g., a gene associated with a particular disease), viral RNA and DNA, bacterial DNA, fungal DNA, cDNA, mRNA, RNA and DNA fragments, oligonucleotides, synthetic oligonucleotides, modified oligonucleotides, single-stranded and double-stranded nucleic acids, natural and synthetic nucleic acids, etc. Thus, examples of the uses of the methods of targeting a nucleic acid include: the diagnosis and/or monitoring of viral diseases (e.g., human immunodeficiency virus, hepatitis viruses, herpes viruses, cytomegalovirus, and Epstein-Barr virus), bacterial diseases (e.g., tuberculosis, Lyme disease, H. pylori, Escherichia coli infections, Legionella infections, Mycoplasma infections, Salmonella infections), sexually transmitted diseases (e.g., gonorrhea), inherited disorders (e.g., cystic fibrosis, Duchenne muscular dystrophy, phenylketonuria, sickle cell anemia), and cancers (e.g., genes associated with the development of cancer); in forensics; in DNA sequencing; for paternity testing; for cell line authentication; for monitoring gene therapy; and for many other purposes.

“Hybridization,” which is used interchangeably herein with the term “complex formation” herein in aspects of the invention wherein the binding agent is an oligonucleotide, means an interaction between two or three strands of nucleic acids by hydrogen bonds in accordance with the rules of Watson-Crick DNA complementarity, Hoogstein binding, or other sequence-specific binding known in the art. Hybridization can be performed under different stringency conditions known in the art.

The contacting of the nanoparticle-oligonucleotide conjugates with the nucleic acid takes place under conditions effective for hybridization of the oligonucleotides on the nanoparticles with the target sequence(s) of the nucleic acid. These hybridization conditions are well known in the art and can readily be optimized for the particular system employed. See, e.g., Sambrook et al., Molecular Cloning: A Laboratory Manual (2nd ed. 1989). Preferably stringent hybridization conditions are employed.

In various aspects, methods include use of two or three oligonucleotides which are 100% complementary to each other, i.e., a perfect match, while in other aspects, the individual oligonucleotides are at least (meaning greater than or equal to) about 95% complementary to each over the all or part of length of each oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to each other. Thus, it will be understood that an oligonucleotide used in the methods needs not be 100% complementary to a desired target nucleic acid to be specifically hybridizable. Moreover, oligonucleotide may hybridize to each other over one or more segments such that intervening or adjacent segments are not involved in the hybridization event (e.g., a loop structure or hairpin structure). Percent complementarity between any given oligonucleotide can be determined routinely using BLAST programs (Basic Local Alignment Search Tools) and PowerBLAST programs known in the art (Altschul et al., 1990, J. Mol. Biol., 215: 403-410; Zhang and Madden, 1997, Genome Res., 7: 649-656).

The target nucleic acid may be isolated by known methods, or may be in cells, tissue samples, biological fluids (e.g., saliva, urine, blood, serum), solutions containing PCR components, solutions containing large excesses of oligonucleotides or high molecular weight DNA, and other samples, as also known in the art. See, e.g., Sambrook et al., Molecular Cloning: A Laboratory Manual (2nd ed. 1989) and B. D. Hames and S. J. Higgins, Eds., Gene Probes 1 (IRL Press, New York, 1995). Methods of preparing nucleic acids for hybridizing with probes are well known in the art. See, e.g., Sambrook et al., Molecular Cloning: A Laboratory Manual (2nd ed. 1989) and B. D. Hames and S. J. Higgins, Eds., Gene Probes 1 (IRL Press, New York, 1995).

If a nucleic acid is present in small amounts, it may be amplified by methods known in the art. See, e.g., Sambrook et al., Molecular Cloning: A Laboratory Manual (2nd ed. 1989) and B. D. Hames and S. J. Higgins, Eds., Gene Probes 1 (IRL Press, New York, 1995). Generally, but without limitation, polymerase chain reaction (PCR) amplification can be performed to increase the concentration of a target nucleic acid to a degree that it can be more easily detected.

Faster hybridization can be obtained by freezing and thawing a solution containing the nucleic acid to be detected and the nanoparticle-oligonucleotide conjugates. The solution may be frozen in any convenient manner, such as placing it in a dry ice-alcohol bath for a sufficient time for the solution to freeze. The solution must be thawed at a temperature below the thermal denaturation temperature, which can conveniently be room temperature for most combinations of nanoparticle-oligonucleotide conjugates and nucleic acids. The hybridization is complete, and the detectable change may be observed, after thawing the solution.

The rate of hybridization can also be increased by warming the solution containing the target nucleic acid and the nanoparticle-oligonucleotide conjugates to a temperature below the dissociation temperature (Tₘ) for the complex formed between the oligonucleotides on the nanoparticles and the target nucleic acid. Alternatively, rapid hybridization can be achieved by heating above the dissociation temperature (Tₘ) and allowing the solution to cool. Alternatively, the rate of hybridization can also be increased by increasing the salt concentration. Importantly, the Ag NP-binding agent conjugates of the invention are highly stable at such conditions.

One method according to the invention for targeting a nucleic acid comprises contacting a target nucleic acid with one or more types of nanoparticles having an oligonucleotide attached thereto. The target nucleic acid to be detected has at least two portions. The lengths of these portions and the distance(s), if any, between the portions are chosen so that when the oligonucleotides on the nanoparticles hybridize to the nucleic acid, a detectable change occurs. These lengths and distances can be determined empirically and will depend on the type of particle used and its size and the type of electrolyte which will be present in solutions used in the assay (as is known in the art, certain electrolytes affect the conformation of nucleic acids).

In some aspects, a specific nucleic acid is targeted using a first oligonucleotide attached to a first nanoparticle has a sequence complementary to a first sequence in the target sequence and a second oligonucleotide attached to a second nanoparticle has a sequence complementary to a second sequence of the target sequence in the DNA. Additional sequences of the target nucleic acid could be targeted with corresponding nanoparticles. Targeting several sequences of a target nucleic acid increases the magnitude of the detectable change.

The oligonucleotide-nanoparticle conjugates that employ cyclic disulfide linkers are, in one aspect, used as probes in diagnostic assays for detecting nucleic acids. These conjugates according to the present invention have unexpectedly been found to improve the sensitivity of diagnostic assays in which they are used. In particular, assays employing oligonucleotide-nanoparticle conjugates prepared using linkers comprising a steroid residue attached to a cyclic disulfide have been found to be about 10 times more sensitive than assays employing conjugates prepared using alkanethiols or acyclic disulfides as the linker.

Some embodiments of the method of detecting target nucleic acid utilize a substrate. By employing a substrate, the detectable change can be amplified and the sensitivity of the assay increased.

In one aspect, the method comprises the steps of contacting a target nucleic acid with a substrate having an oligonucleotide attached thereto, the oligonucleotide (i) having a sequence complementary to a first portion of the sequence of the target nucleic acid, the contacting step performed under conditions effective to allow hybridization of the oligonucleotide on the substrate with the target nucleic acid, and (ii) contacting the target nucleic acid bound to the substrate with a first type of nanoparticle having an oligonucleotide attached thereto, the oligonucleotide having a sequence complementary to a second portion of the sequence of the target nucleic acid, the contacting step performed under conditions effective to allow hybridization of the oligonucleotide on the nanoparticles with the target nucleic acid. Next, the first type of nanoparticle-oligonucleotide conjugate bound to the substrate is contacted with a second type of nanoparticle having an oligonucleotide attached thereto, the oligonucleotide on the second type of nanoparticle having a sequence complementary to at least a portion of the sequence of the oligonucleotide on the first type of nanoparticle, the contacting step taking place under conditions effective to allow hybridization of the oligonucleotides on the first and second types of nanoparticles. Finally, a detectable change produced by these hybridizations is observed.

Any substrate can be used which allows observation of the detectable change. Suitable substrates include transparent solid surfaces (e.g., glass, quartz, plastics and other polymers), opaque solid surface (e.g., white solid surfaces, such as TLC silica plates, filter paper, glass fiber filters, cellulose nitrate membranes, nylon membranes), and conducting solid surfaces (e.g., indium-tin-oxide (ITO)). The substrate can be any shape or thickness, but generally will be flat and thin. Preferred are transparent substrates such as glass (e.g., glass slides) or plastics (e.g., wells of microtiter plates). Method of attaching oligonucleotides to a substrate and uses thereof with respect to nanoparticle-binding agent conjugates are disclosed in U.S. Patent Application 20020172953, incorporated herein by reference in its entirety.

The detectable change that occurs upon hybridization of the oligonucleotides on the nanoparticles to the nucleic acid may be a color change, the formation of aggregates of the nanoparticles, or the precipitation of the aggregated nanoparticles. The color changes can be observed with the naked eye or spectroscopically. The formation of aggregates of the nanoparticles can be observed by electron microscopy or by nephelometry. The precipitation of the aggregated nanoparticles can be observed with the naked eye or microscopically. Preferred are changes observable with the naked eye. Particularly preferred is a color change observable with the naked eye.

The methods of detecting target nucleic acids hybridization based on observing a color change with the naked eye are cheap, fast, simple, robust (the reagents are stable), do not require specialized or expensive equipment, and little or no instrumentation is required. This makes them particularly suitable for use in, e.g., research and analytical laboratories in DNA sequencing, in the field to detect the presence of specific pathogens, in the doctor's office for quick identification of an infection to assist in prescribing a drug for treatment, and in homes and health centers for inexpensive first-line screening.

In various embodiments, the target nucleic acid is a mRNA encoding a gene product and translation of the gene product is inhibited, or the target nucleic acid is DNA in a gene encoding a gene product and transcription of the gene product is inhibited. In methods wherein the target nucleic acid is DNA, the polynucleotide is in certain aspects DNA which encodes the gene product being inhibited. In other methods, the DNA is complementary to a coding region for the gene product. In still other aspects, the DNA encodes a regulatory element necessary for expression of the gene product. “Regulatory elements” include, but are not limited to enhancers, promoters, silencers, polyadenylation signals, regulatory protein binding elements, regulatory introns, ribosome entry sites, and the like. In still another aspect, the target nucleic acid is a sequence which is required for endogenous replication.

The terms “start codon region” and “translation initiation codon region” refer to a portion of a mRNA or gene that encompasses contiguous nucleotides in either direction (i.e., 5′ or 3′) from a translation initiation codon. Similarly, the terms “stop codon region” and “translation termination codon region” refer to a portion of such a mRNA or gene that encompasses contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the oligonucleotides on the functionalized nanoparticles.

Other target regions include the 5′ untranslated region (5′UTR), the portion of an mRNA in the 5′ direction from the translation initiation codon, including nucleotides between the 5′ cap site and the translation initiation codon of a mRNA (or corresponding nucleotides on the gene), and the 3′ untranslated region (3′UTR), the portion of a mRNA in the 3′ direction from the translation termination codon, including nucleotides between the translation termination codon and 3′ end of a mRNA (or corresponding nucleotides on the gene). The 5′ cap site of a mRNA comprises an N7-methylated guanosine residue joined to the 5′-most residue of the mRNA via a 5′-5′ triphosphate linkage. The 5′ cap region of a mRNA is considered to include the 5′ cap structure itself as well as the first 50 nucleotides adjacent to the cap site.

For prokaryotic target nucleic acid, in various aspects, the nucleic acid is genomic DNA or RNA transcribed from genomic DNA. For eukaryotic target nucleic acid, the nucleic acid is an animal nucleic acid, a plant nucleic acid, a fungal nucleic acid, including yeast nucleic acid. As above, the target nucleic acid is either a genomic DNA or RNA transcribed from a genomic DNA sequence. In certain aspects, the target nucleic acid is a mitochondrial nucleic acid. For viral target nucleic acid, the nucleic acid is viral genomic RNA, viral genomic DNA, or RNA transcribed from viral genomic DNA.

Methods for inhibiting gene product expression provided include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of an oligonucleotide-functionalized nanoparticle. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of nanoparticle and a specific oligonucleotide.

A probe oligonucleotide is an oligonucleotide used in an assay to target an analyte of interest. The probe oligonucleotide can be used in an assay such as a bio barcode assay. See, e.g., U.S. Pat. Nos. 6,361,944; 6,417,340; 6,495,324; 6,506,564; 6,582,921; 6,602,669; 6,610,491; 6,678,548; 6,677,122; 6,682,895; 6,709,825; 6,720,147; 6,720,411; 6,750,016; 6,759,199; 6,767,702; 6,773,884; 6,777,186; 6,812,334; 6,818,753; 6,828,432; 6,827,979; 6,861,221; and 6,878,814, the disclosures of which are incorporated herein by reference.

### Polypeptides

As used herein, the term “polypeptide” when used as a binding agent or a target molecule refers to peptides, proteins, polymers of amino acids, hormones, viruses, and antibodies that are naturally derived, synthetically produced, or recombinantly produced. Polypeptides also include lipoproteins and post-translationally modified proteins, such as, for example, glycosylated proteins, as well as proteins or protein substances that have D-amino acids, modified, derivatized, or non-naturally occurring amino acids in the D- or L-configuration and/or peptomimetic units as part of their structure.

Accordingly, targeting agents contemplated include nuclear localization signals (NLS) and peptide transduction domains, including, for example, SV40 large T NLS, HIV-1 TAT protein NLS, adenovirus NLS, integrin binding domain, oligolysine (each of which is described in (Tkachenko, et al., 2004, Bioconjugate Chem 15:482-490), and adenovirus fiber protein comprising both NLS and receptor-mediated endocytosis (RME) domains (Tkachenko, et al., 2003, Am. Chem. Soc. 125:4700-4701).

In other embodiments of the methods, the nanoparticle-binding agent conjugate is selected based on its binding specificity to a ligand expressed in or on a target cell type or a target organ. Alternatively, conjugates of this type include a receptor for a ligand on a target cell (instead of the ligand itself), and in still other embodiments, both a receptor and its ligand are contemplated in those instances wherein a target cell expresses both the receptor and the ligand. In other embodiments, members from this group are selected based on their biological activity, including for example enzymatic activity, agonist properties, antagonist properties, multimerization capacity (including homo-multimers and hetero-multimers). With regard to proteins, conjugate binding agent contemplated include full length protein and fragments thereof which retain the desired property of the full length proteins. Fusion proteins, including fusion proteins wherein one fusion component is a fragment or a mimetic, are also contemplated. This group also includes antibodies along with fragments and derivatives thereof, including but not limited to Fab′ fragments, F(ab)₂ fragments, Fv fragments, Fc fragments, one or more complementarity determining regions (CDR) fragments, individual heavy chains, individual light chain, dimeric heavy and light chains (as opposed to heterotetrameric heavy and light chains found in an intact antibody, single chain antibodies (scAb), humanized antibodies (as well as antibodies modified in the manner of humanized antibodies but with the resulting antibody more closely resembling an antibody in a non-human species), chelating recombinant antibodies (CRABs), bispecific antibodies and multispecific antibodies, and other antibody derivative or fragments known in the art.

In some aspects, the binding agent provides a functional group that is subject to cleavage by an enzyme or other catalyst or hydrolytic conditions found in the target tissue or organ or cell.

### Nanofabrication

The invention also provides a method of using the silver nanoparticles described herein as synthons for nanofabrication, also referred to as programmable materials synthesis (Storhoff et al., 1999 Chem. Rev., 99 (7), 1849-1862). A “synthon” as used herein is understood to mean a template. The method comprises providing at least one type of linking oligonucleotide having a selected sequence. “Linking oligonucleotides” according to the invention are characterized herein below.

A linking oligonucleotide used for nanofabrication may have any desired sequence and may be single-stranded or double-stranded. It may also contain chemical modifications in the base, sugar, or backbone sections as described herein. The sequence chosen for the linking oligonucleotide, their lengths and strandedness will contribute to the rigidity or flexibility of the resulting nanomaterial or nanostructure, or a portion of the nanomaterial or nanostructure. The use of a single type of linking oligonucleotide, as well as mixtures of two or more different types of linking oligonucleotides, is contemplated. The number of different linking oligonucleotides used and their lengths will contribute to the shapes, pore sizes and other structural features of the resulting nanomaterials and nanostructures.

The sequence of a linking oligonucleotide will have at least a first portion and a second portion for binding to oligonucleotides on nanoparticles. The first, second or more binding portions of the linking oligonucleotide may have the same or different sequences.

If all of the binding portions of a linking oligonucleotide have the same sequence, only a single type of nanoparticle with oligonucleotides having a complementary sequence attached thereto need be used to form a nanomaterial or nanostructure. If the two or-more binding portions of a linking oligonucleotide have different sequences, then two or more nanoparticle-oligonucleotide conjugates must be used. The oligonucleotides on each of the nanoparticles will have a sequence complementary to one of the two or more binding portions of the sequence of the linking oligonucleotide. The number, sequence(s) and length(s) of the binding portions and the distance(s), if any, between them will contribute to the structural and physical properties of the resulting nanomaterials and nanostructures. Of course, if the linking oligonucleotide comprises two or more portions, the sequences of the binding portions must be chosen so that they are not complementary to each other to avoid having one portion of the linking nucleotide bind to another portion.

The linking oligonucleotides and nanoparticle-oligonucleotide conjugates are contacted under conditions effective for hybridization of the oligonucleotides attached to the nanoparticles with the linking oligonucleotides so that a desired nanomaterial or nanostructure is formed wherein the nanoparticles are held together by oligonucleotide connectors. These hybridization conditions are well known in the art and can be optimized for a particular nanofabrication scheme as discussed herein. Stringent hybridization conditions are preferred.

The invention also provides additional method of nanofabrication. One such method comprises providing at least two types of nanoparticles having oligonucleotides attached thereto. An oligonucleotide on the first type of nanoparticles has a sequence complementary to that of an oligonucleotide on a second type of nanoparticle. The first and second types of nanoparticles are contacted under conditions effective to allow hybridization of the oligonucleotides on the nanoparticles to each other so that a desired nanomaterials or nanostructure is formed.

In both nanofabrication methods of the invention, the use of nanoparticles having one or more different types of oligonucleotides attached thereto is contemplated. The number of different oligonucleotides attached to a nanoparticle and the lengths and sequences of the one or more oligonucleotides will contribute to the rigidity and structural features of the resulting nanomaterials and nanostructures.

Also, as discussed above for functionalized nanoparticles in general, the size, shape and chemical composition of the nanoparticles will contribute to the properties of the resulting nanomaterials and nanostructures. These properties include optical properties, optoelectronic properties, electrochemical properties, electronic properties, stability in various solutions, pore and channel size variation, ability to separate bioactive molecules while acting as a filter, etc. The use of mixtures of nanoparticles having different sizes, shapes and/or chemical compositions, as well as the use of nanoparticles having uniform sizes, shapes and chemical composition, are contemplated.

Nanofabrication methods of the invention are extremely versatile. By varying the length, sequence and strandedness of the linking oligonucleotide, the number, length, and sequence of the binding portions of the linking oligonucleotide, the length, sequence and number of the oligonucleotides attached to the nanoparticles, the size, shape and chemical composition of the nanoparticles, the number and types of different linking oligonucleotides and nanoparticles used, and the strandedness of the oligonucleotide connectors, nanomaterials and nanostructures having a wide range of structures and properties can be prepared. These structures and properties can be varied further by cross-linking of the oligonucleotide connectors, by functionalizing the oligonucleotides, by backbone, base or sugar modifications of the oligonucleotides, or by the use of peptide-nucleic acids.

The nanomaterials and nanostructures that can be made by the nanofabrication methods of the invention include nanoscale mechanical devices, separation membranes, bio-filters, and biochips. It is contemplated that the nanomaterials and nanostructures of the invention can be used as chemical sensors, in computers, for drug delivery, for protein engineering, and as templates for biosynthesis/nanostructure fabrication/directed assembly of other structures. See generally Seeman et al., 1993 New J. Chem., 17, 739 for other possible applications. The nanomaterials and nanostructures that can be made by the nanofabrication method of the invention also can include nanoelectronic devices. U.S. Pat. No. 6,767,702 demonstrated that nanoparticles assembled by DNA possess the ability to conduct electricity (the DNA connectors function as semiconductors).

Additional methods of nanofabrication are generally disclosed in U.S. Patent Application 20020172953.

The invention is illustrated by the following examples, which are not intended to be limiting in any way.

### EXAMPLES

### Example 1

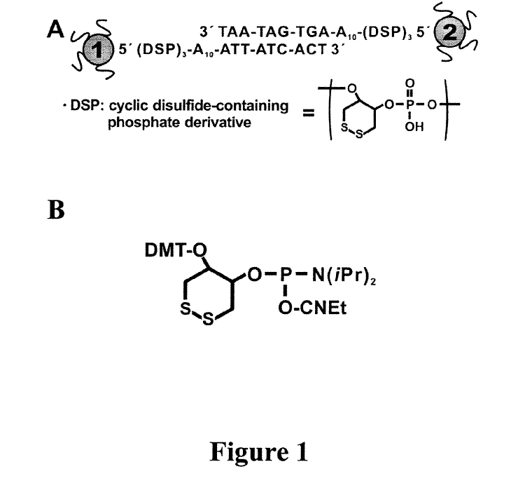

One OD of an oligonucleotide modified with three cyclic disulfide units (1: 5′ (DSP)₃-A₁₀-ATT-ATC-ACT 3′ (SEQ ID NO. 1); 2: 5′ (DSP)₃-A₁₀-AGT-GAT-AAT 3′ (SEQ ID NO. 2); DSP: cyclic disulfide-containing phosphate derivative; see FIG. 1A) was added to 1 mL of Ag NP (30 nm in diameter, 1.2 nM) solution. Silver nanoparticles were purchased from Ted Pella. The oligonucleotides were prepared through solid-phase syntheses on 1 μmol scales using controlled pore glass beads (CPG) and standard phosphoramidite chemistry on an automated synthesizer (Milligene Expedite). For the 5′ terminal modification of oligonucleotides with cyclic disulfide anchors, three disulfide-containing phosphoramidite units (DTPA; Glen Research, Cat. No. 10-1937-90, FIG. 1B) were coupled in a series on the synthesizer with an extended coupling time (10 min) each. DTPA becomes DSP through the synthesis cycles. The synthesized oligonucleotides were cleaved from the CPG support by incubation in 1.5 mL of NH₄OH (30% v/v) for 16 h at 56° C. After the removal of ammonia under N₂ flow, the crude product was collected, filtered with 0.2 μm cellulose acetate (CA) syringe filter (Whatman), and purified by reverse-phase HPLC on a Hewlett-Packard Series 1100 system (10×250 mm Varian DYNAMAX C18 column). After HPLC purification, the protecting dimethoxytrityl (DMT) groups at 5′ termini of oligonucleotides were removed by incubating in 70% glacial acetic acid aqueous solution for 30 min at room temperature followed by lyophilization to get dry product. The dry product was dissolved in water again, and the detached DMT was extracted from aqueous solution using ethyl acetate. The purified DNA was aliquoted, lyophilized and stored in a freezer (−20° C.). The final oligonucleotide concentration was ˜4.7 μM, and the final Ag NP concentration was ˜1 nM (ε_{410 nm}=1.2×10¹⁰ cm⁻¹M⁻¹) (Li et al., Nucleic Acids Res. 2002, 30, 1558-1562).

This step was followed by the addition of 1% sodium dodecyl sulfate (SDS) aqueous solution (final concentration=0.01% SDS) and 100 mM phosphate buffer (final concentration=10 mM phosphate, pH 7.4). Over a period of 30 min, 2 M NaCl solution was added in a stepwise manner (final concentration=0.15 M NaCl). The solution was incubated overnight at room temperature, followed by centrifugation to isolate the particles. The supernatant was removed, and the particles were redispersed in phosphate buffer (0.01% Tween 20, 10 mM phosphate, a desired concentration of NaCl, pH 7.4). This step was repeated three times to eliminate residual DNA. Equal concentrations of Ag NPs modified with SEQ ID NO. 1 or SEQ ID NO. 2 were combined and allowed to hybridize at room temperature.

These DNA-functionalized silver particles (DNA-Ag NPs) can be synthesized in less than 30 minutes and show stability at high salt concentrations (1.0 M NaCl) (see below). Significantly, the DNA-Ag NPs also exhibit high cooperativity as characterized by their sharp DNA melting transitions (full width at half-maximum (FWHM)=˜2° C.) (see below).

In order to study the properties of DNA-Ag NPs, two batches of silver nanoparticles were functionalized with complementary oligonucleotide sequences (sequence 1 and 2, respectively) (SEQ ID NOs 1 and 2), FIG. 1A. Unmodified silver nanoparticles exhibit a surface plasmon resonance at 410 nm, and therefore they exhibit an intense yellow color. Interestingly, these particles do not show any noticeable changes in their UV-vis spectrum after DNA-functionalization, indicating that the particles are stable and do not aggregate. This has been confirmed by TEM analysis of the modified particles. However, when DNA-Ag NPs, modified with complementary sequences 1 and 2 (SEQ ID NOs 1 and 2), respectively, are combined, the plasmon resonance dampens and red-shifts from 410 to 560 nm. This dampening and red-shifting is a result of particle assembly due to hybridization, which can be observed with the naked eye in the form of a color change from bright yellow to pale red. Since the process is due to DNA hybridization, it is reversible, and upon heating, the color of the solution returns to an intense yellow, a diagnostic indicator of dehybridization in this system.

### Example 2

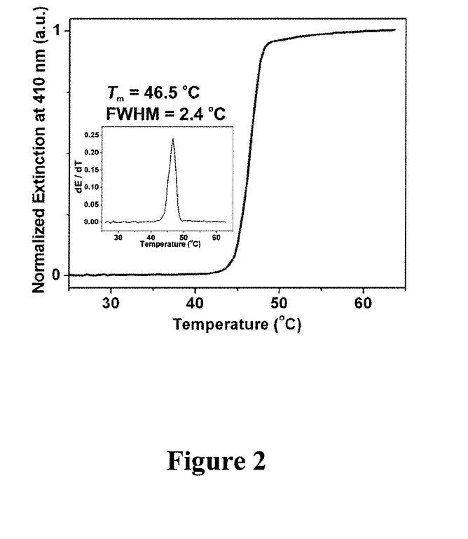

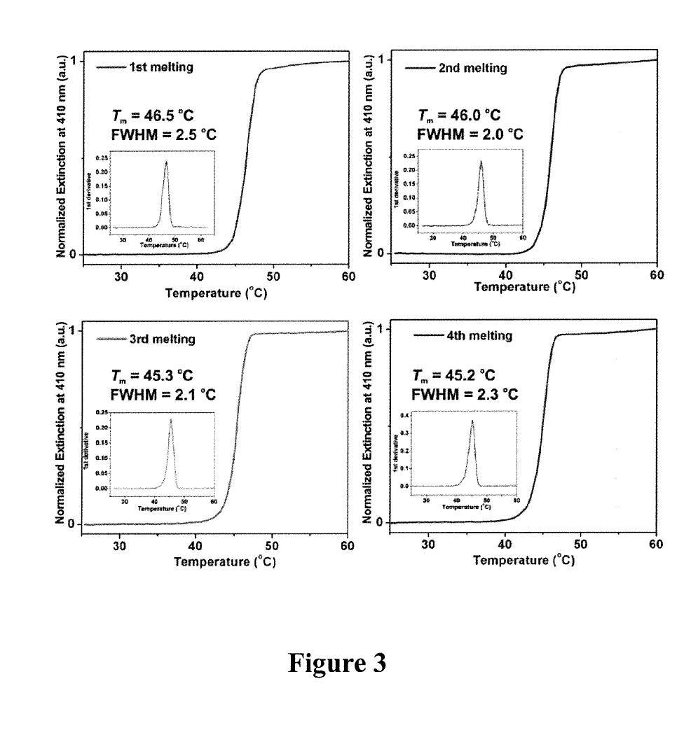

The reversible nature of the DNA-Ag NP hybridization process was further characterized by monitoring the melting process at 410 nm as a function of temperature (FIG. 2). Importantly, the DNA-linked Ag NPs exhibit a sharp melting transition similar to that characteristic of the analogous DNA-linked Au NP aggregates (Jin et al., J. Am. Chem. Soc. 2003, 125, 1643-1654), indicating that DNA-linked Ag NPs also exhibit highly cooperative binding properties. The melting temperature (Tₘ) of the DNA-linked Ag NPs, 46.5° C., was obtained by taking the maximum of the first derivative of the melting profile. The FWHM of the first derivative (FIG. 2, inset) is ˜2.4° C., which is comparable to the typical sharp melting transition of DNA-linked Au NPs (FWHM=˜2.2° C.) (Storhoff et al., J. Am. Chem. Soc. 1998, 120, 1959-1964). Significantly, this melting transition was found to be highly reproducible, as demonstrated by repeated hybridization/melting experiments, which were performed with the same sample of the DNA-Ag NPs over a period of one week (FIG. 3). Melting transitions of hybridized DNA-Ag NPs were analyzed by monitoring the change in extinction at 410 nm at 0.5° C. interval at a rate of 1° C./min (Cary 5000 equipped with a Peltier temperature controller, Varian). The concentration of total DNA-Ag NPs was nM, and the concentration of NaCl was 0.15 M.

This reproducibility is strong evidence that the modification of the Ag NP surface with oligonucleotides through a triple cyclic disulfide anchor is strong enough to stabilize the DNA-Ag NP probes against heat, aging, and degradation in aqueous media.

### Example 3

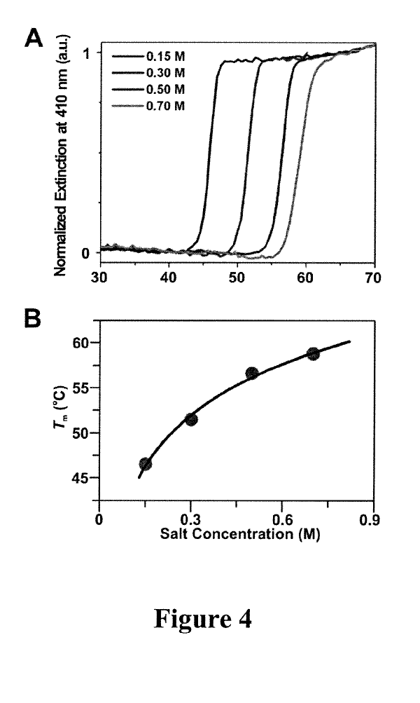

To determine the effect of the salt concentration on the melting properties of DNA-Ag NP aggregates, the melting transitions of DNA-Ag NP aggregates as a function of NaCl concentration was monitored. As expected, the melting transitions occur at higher temperatures as the salt concentration increases (FIG. 4A) due to enhanced screening, which decreases the repulsion between the negatively charged oligonucleotides, as previously reported with DNA-Au NPs (Jin et al., J. Am. Chem. Soc. 2003, 125, 1643-1654). The Tₘ spans the range from 46.5° C. to 58.8° C., as the salt concentration is increased from 0.15 M to 0.70 M (FIG. 4B). Note that the functionalized Ag NPs are stable at high salt concentrations (up to 1.0 M NaCl). Importantly, all of the melting transitions are extremely sharp over the entire salt concentration range studied (FWHM≦˜2.5° C.). This observation demonstrates that the DNA-Ag NP hybridization and dehybridization process can be controlled by adjusting salt concentrations similar to the control afforded by DNA-Au NPs.

What is claimed:

1. A silver nanoparticle-binding agent conjugate comprising one or more binding agents attached covalently to a silver nanoparticle through a triple cyclic disulfide functional group moiety.

2. The nanoparticle of claim 1 wherein the binding agent is an oligonucleotide.

3. The nanoparticle of claim 2 wherein the oligonucleotides comprises a sequence complementary to at least one portion of a sequence of a target nucleic acid.

4. The nanoparticle of claim 3 wherein in the presence of the nucleic acid target and under appropriate hybridization conditions, the oligonucleotide attached to the nanoparticle forms an oligonucleotide/nucleic acid target complex with the nucleic acid target.

5. The nanoparticle of claim 4 wherein the oligonucleotide/nucleic acid target complex has a sharper melting profile and higher stability, relative to a melting profile and stability of an analogous complex formed between the nucleic acid target and a free oligonucleotide having a sequence identical to the sequence of an oligonucleotide bound to the nanoparticle.

6. The nanoparticle of claim 1 wherein the binding agent is an antibody.

7. The nanoparticle of claim 1 wherein the binding agent is a peptide.

8. The nanoparticle according to claim 1 wherein the diameter of said nanoparticle is between about 10 nm and about 100 nm, inclusive.

9. The nanoparticle according to claim 1 wherein the triple cyclic disulfide functional group moiety comprises a cyclic disulfide-containing phosphate derivative.

10. The nanoparticle according to claim 1 further comprising one or more additional cyclic disulfide functional group moieties.

11. The nanoparticle according to claim 1 wherein the binding agent comprises a spacer portion and a binding portion, the spacer portion being bound to the nanoparticle and comprising the triple cyclic disulfide functional group moiety through which the binding agent is bound to the nanoparticle.

12. The nanoparticle according to claim 11 wherein the spacer portion comprises from about 1 monomer subunits to about 10 monomer subunits, inclusive.

13. The nanoparticle according to claim 11 wherein the monomer subunits are nucleotides.

14. The nanoparticle according to claim 13 wherein the spacer portion comprises from about 10 to about 30 nucleotides, inclusive.

15. The nanoparticle according to claim 13 wherein the spacer portion comprises all adenine bases, all thymine bases, all cytosine bases, all uracil bases or all guanine bases.

16. A method of attaching a binding agent to a silver nanoparticle to produce a nanoparticle-binding agent conjugate, the method comprising:
contacting (i) a binding agent having a triple cyclic disulfide functional group moiety covalently bound thereto, and (ii) a silver nanoparticle under conditions effective to allow the binding agent to attach to the nanoparticle through the triple cyclic disulfide functional group moiety to produce the nanoparticle-binding agent conjugate.

17. The method according to claim 16 wherein at least two binding agents are attached to the nanoparticle wherein the two binding agents bind to different target binding partners.

18. The method according to claim 17 wherein a binding agent attached to the nanoparticle has the ability to bind under appropriate conditions to a target binding partner attached to a second nanoparticle to form an aggregate.
